(19) United States
(12) Patent Application Publication
(10) Pub. No.: US 2010/0130890 A1
(43) Pub. Date: May 27, 2010

(54) ACTIVITY MEASUREMENT SYSTEM

(76) Inventors: Yoshihiro Matsumura, Kadoma-shi (JP); Kenji Nishino, Ibaraki-shi (JP); Yutaka Yamanaka, Hirakata-shi (JP); Tadaharu Kitadou, Moriguchi-shi (JP); Masayuki Naruo, Hirakata-shi (JP)

Correspondence Address: Cheng Law Group, PLLC, 1100 17th Street, N.W., Suite 503, Washington, DC, 20036, US

(21) Appl. No.: 12/593,992

(22) PCT Filed: Mar. 27, 2008
(86) PCT No.: PCT/JP2008/055908

§ 371 Date: Sep. 30, 2009

(30) Foreign Application Priority Data
Mar. 30, 2007 (JP) 2007-095268

(51) Int. Cl. A61B 5/11

(52) U.S. Cl. 600/595

(57) ABSTRACT

An activity measurement system gives, based upon consecutively measured activity intensities, an exercise index (EX) indicative of energetic exercise and a daily activity index (DA) indicative of a routine living activity within a predetermined measurement time period, and display an activity element A (x, y), a combination of these indexes within an X-Y matrix plane. The exercise index (EX) is calculated at a first operation means as a sum of a product of the activity intensity exceeds a predetermined reference and a time period during which the activity intensity exceeds the reference, within the measurement time period. The daily activity index (DA) is calculated at a second operation means as a caloric consumption divided by a basal metabolic rate of a user within the measurement time period. The caloric consumption is determined a function of the activity intensity per unit time below the reference and data indicative of physical characteristics of the user. The basal metabolic rate is calculated based upon the data of the physical characteristics which are stored in a personal information data table provided in the system.

### TECHNICAL FIELD

The present invention is directed to an activity measurement system which measures a user's activity for displaying the same.

### BACKGROUND ART

JP2003-024287 A discloses an activity measurement system for evaluation of an exercise by use of an acceleration sensor. The system is designed to measure activity intensity with regard to an exercise made by a user, and is not concerned about the activity with regard to a routine living activity. However, in view of that the user's activity involves energetic exercise activity and the routine living activity; it is desired to give a comprehensive evaluation of these activities.

### DISCLOSURE OF THE INVENTION

The present invention has been achieved to solve the above problem and has an object of providing an activity measurement system which is capable of displaying the activity indicative of energetic exercise in contrast to the activity resulting from the routine living activity. The activity measurement system in accordance with the present invention includes an activity detection means configured to detect an activity of a user and obtain an activity intensity per unit time with regard to the activity, a first operation means configured to obtain a first index from the activity intensity, and a second operation means configured to obtain, from the activity intensity, a second index which is different from the first index. The system further includes a personal information table storing physical characteristics of a user, and a display means. The display means includes a display presenting an X-Y matrix plane and is configured to show an activity information indication which is a combination of the first and second indexes at a corresponding position in a coordinate system defined by the fist and second indexes. The first operation means is configured to obtain the first index which is sum of products of the activity intensity exceeding a predetermined intensity and a time during which the activity intensity exceeds the reference intensity within a predetermined measurement time period. The second operation means is configured to obtain a total of a consumption energy defined by a function of the activity intensity per unit time and the physical characteristics of the user read out from the personal information table within the measurement time period, to obtain, based on the physical characteristics of the user read out from the personal information table, a basal metabolic rate required within the measurement time period, and to provide the second index which is the consumption energy divided by the basal metabolic rate. Accordingly, the first index indicates the activity resulting from energetic exercise, while the second index indicates the activity resulting from the routine living activity not relating to the energetic exercise. With the presentation of these two activities in the matrix plane, it is possible to display a trend of the user's activity. Particularly, as the second index is defined by a quotient of the consumption energy consumed by the activity during the measurement time period divided by the sum of the basal metabolic rate during the same measurement time period, the user's routine living activity can be displayed.

Preferably, the second operation means is configured to obtain the consumption energy with reference to the activity intensity only below the reference intensity. Thus, the activity resulting from the routine living activity can be specified as excluding the energetic exercise so as to make a definite contrast with the first index designating the energetic exercise.

Generally, the user's behavior is expected to be defined weekly, the measurement time period is preferably set to be a week.

Further, the reference intensity is preferred to vary with the user's age and sex. For this purpose, the system has a reference memory storing different reference intensities in association with the user's age and sex, and the second operation means is configured to select the reference intensity according to the user's age and sex. With this arrangement, the system can exactly distinct the routine living activity from the energetic activity in view of the user's age and the sex.

The system can be realized in a portable terminal carried by the user. In this instance, a terminal case carried to the user is equipped with the activity detection means, the first operation means, the second operation means, and the display means.

Alternatively, the system can be realized by a portable terminal and a server that transmits and receives data to and from the portable terminal. In this instance, the portable terminal is equipped with the activity detection means, while the server is equipped with the first operation means, the second operation means, and the display means.

Further, the system may be realized by a plurality of portable terminals carried by different users, and a server. In this instance, the server is provided with an activity data table configured to store the first index and the second index obtained by each of the portable terminals, and a user data table configured to store attributes of the users. The server is also equipped with a server-side display means having a display having a X-Y matrix plane. The server-side display means is configured to provide, in the X-Y matrix, the activity information indication transmitted from a particular one of the portable terminals, as well as to provide the activity information indication given for the other users classified in the same user group. With this result, the user is enabled to compare one's own activity information with the other users in the same classification so as to be given a motivation for executing one's own exercise project.

The present system is preferred to display, in addition to magnitude of the activity, kinds of particular predetermined exercises and particular living activities associated with the activity intensities, in contrast to the first and second indexes. In this instance, the activity detection means includes an acceleration sensor, an activity intensity calculation means, and a footstep counter means. The acceleration sensor is configured to output the acceleration data generated according to the user's activity. The activity intensity calculation means is configured to determine, based upon the acceleration data, the activity intensity per unit time. The footstep counter means is configured to count the number of footsteps per unit time based upon the acceleration data. The system further includes an activity history table that stores a time series data of the activity intensity and the number of the footsteps per unit time in association with a measurement time, an activity classification table that classifies the kinds of the activity in terms of the activity intensity and the number of footsteps as parameters; and an activity classifying means that analyzes the time-series data of the activity intensity and the number of footsteps at each measurement time and compare the same from those of the activity classification table so as to determine similarity of the data with one of the kinds of the activity in the activity classification table, to identify the kind of the activity at each measurement time based upon the similarity, and to obtain a ratio of each identified kind of the activity within the measurement time period. Thus obtained ratio of the kinds of the activity is displayed in the matrix plane of the display means together with a content indicative of the kind of the activity shown along the coordinate axes of the matrix plane. Accordingly, the trend of the activity during the measurement time period is presented in association with the kinds of the activity, thereby giving more exact analysis result of the activity to the user.

Preferably, the cluster analysis is utilized as one analyzing scheme for identification of the kind of the activity and employs parameters of an average activity intensity, a maximum activity intensity, a minimum activity intensity, an average number of the footsteps, a maximum foot-pitch, and a minimum foot pitch included in the above time series data.

Further, the activity measurement system of the present invention is preferred to have a daily schedule table that holds a daily behavior schedule of the user in order to precisely identify the kind of the activity. In this instance, the above activity classifying means is configured to judge whether or not the kind of the activity obtained for each day within the measurement time period is in match with the kind of the activity expected in the daily behavior schedule corresponding to that day, and ignore the kind of the activity not expected in that day. Consequently, an erroneous identification of the kind of the activity is avoided, for example, the kind of exercise not scheduled in weekdays can be ignored in the weekdays.

The activity measurement system of the present invention proposes a scheme in which the activity detection means obtains the activity intensity and the resulting activity with regard to the energetic exercise and routing living activity. The activity detection means includes an acceleration sensor, an activity intensity calculator means, and an equation selector means. The acceleration sensor is configured to output acceleration data generated by the activity of the user. The activity intensity calculator means is configured to obtain the activity intensity by use of a particular equation which is a function of the acceleration data. The equation selector means has an equation table holding a plurality of different equations respectively associated with the different acceleration data, and is configured to retrieve, from the equation table, the equation corresponding to the acceleration data detected at the acceleration sensor, and provide the retrieved equation to the activity intensity calculator means. Accordingly, an optimum equation can be employed in accordance with different accelerations in walking and running to give a reliable activity intensity in well reflection of the kind of the activity for obtaining exact activity in proportion to the activity intensity.

As an alternative to the above scheme, it is possible to select the equation depending on the number of the footsteps derived from the output of the acceleration sensor. In this instance, the activity detection means includes a footstep counter means that determines the number of footsteps per unit time from the acceleration data. The equation selector means has an equation table holding a plurality of different equations respectively associated with the different number of the footsteps, and is configured to retrieve, from the equation table, the equation corresponding to the number of the footsteps detected at the footstep counter means, and provide the retrieved equation to the activity intensity calculator means.

Further, it is possible to combine the above two schemes for obtaining the activity intensity and the activity more precisely. In this instance, the equation selector means is configured to have an equation table holding a plurality of different equations respectively associated with the different number of the footsteps, and also with the different acceleration data, and is configured to retrieve, from the equation table, the equation corresponding to the number of the footsteps detected at the footstep counter means as well as to the acceleration data detected at the acceleration sensor, and provide the retrieved equation to the activity intensity calculator means only when such equation is found to correspond to the number of the footsteps and at the same time to the acceleration data.

Further, the activity measurement system of the present invention proposes a scheme of improving a processing speed of calculating the activity intensity. The activity detection means includes an A/D converter that converts the output from the acceleration sensor into a digital data defined by a predetermined bit array. The activity intensity calculator means is configured to assign the different equations to different partial bit series which are different from each other within the digital data, to extract, from the digital data, the partial bit series corresponding to the equation selected at the equation selector means, and to calculate the activity intensity by use of a numerical value expressed by the partial bit series. With this result, it is made to use an upper bit series for calculation of the activity intensity when the acceleration is relatively large, for example, in running, and to use a lower bit series when the acceleration is relatively small, e.g. in walking, yet providing the same calculation results by use of the whole bit series, thereby improving the calculation speed.

Further, the activity measurement system of the present invention is preferred to have an additional function of displaying the less activity in terms of a ratio of the time within the predetermined measurement time period. For this purpose, the activity detection means includes an acceleration sensor, an activity intensity calculator means, and a footstep counter means. The acceleration sensor is configured to output a time series of acceleration data generated by the activity of the user. The activity intensity calculator means is configured to obtain, from the time series acceleration data, the activity intensity at predetermined intervals by use of a particular equation. The footstep counter means is configured to determine, from the time series of the acceleration data, the number of footsteps per predetermined unit time. In addition, the system includes a low intensity ratio calculator means configured to obtain, within the predetermined measurement time period, a low exercise time period in which the number of footsteps is below a predetermined reference and at the same time the activity intensity is within a predetermined reference range, and to provide a ratio of the low exercise time period to the measurement time period, allowing said display means to display said ratio. With the indication of the ratio of the low exercises time period, the user can be notified of necessity of making exercise.

Furthermore, the activity measurement system of the present invention is preferred to be added with a function of displaying a variation in caloric consumption in combination with a variation in basal metabolic rate occurring in a predetermined judgment time period, based on the measured activity and the basal metabolic rate inherent to the user. In this instance, the activity detection means includes an acceleration sensor, and an activity intensity calculator means. The acceleration sensor is configured to output a time series of acceleration data generated by the activity of the user. The activity intensity calculator means is configured to obtain, from the time series acceleration data, the activity intensity at predetermined intervals by use of a particular equation. The system further includes a user table, a caloric consumption calculator means, and a caloric balance judgment means. The user table holds records of individual information including age, physical characteristics, and fat judgment data specific to the user. The caloric consumption calculator means is configured to obtain a basal metabolic rate based on the age and the physical characteristics, and calculate a caloric consumption based upon thus obtained basal metabolic rate and the activity intensity within a predetermined judgment time period. The caloric balance judgment means is configured to obtain the variation of the caloric consumption as well as the variation of said fat judgment data within the judgment time period so as to give a judgment result in terms of a combination of the variations, which is displayed at the display means. Accordingly, the present system presents the judgment result for notifying the user of the body fat regularly at every judgment time period and prompting to improve diet.

### BRIEF DESCRIPTION OF THE DRAWINGS

FIG. 1 is a schematic perspective-view diagram illustrating an activity measurement system according to an embodiment of the present invention;

FIG. 2 is a block diagram illustrating the internal configuration of the system;

FIG. 3 is a block diagram illustrating a method for obtaining activity intensity, based on acceleration, in the system;

FIG. 4 is an explanatory diagram illustrating an activity data table used in the system;

FIG. 5 is an explanatory diagram illustrating a personal information table used in the system;

FIG. 6 is an explanatory diagram illustrating display content in a matrix plane used in the system;

FIG. 7 is an explanatory diagram illustrating the configuration of the matrix plane;

FIG. 8 is an explanatory diagram illustrating an activity index history table used in the system;

FIG. 9 is an explanatory diagram illustrating an activity history table used in the system;

FIG. 10 is an explanatory diagram illustrating the content displayed in the matrix plane of a server display means in the system;

FIG. 11 is a flow diagram illustrating a method for deciding various advice content to be displayed on the server display means;

FIG. 12 is an explanatory diagram illustrating other display content on the server display means;

FIG. 13 is an explanatory diagram illustrating an activity classification table used in the system;

FIG. 14 is an explanatory diagram illustrating an input screen of a personal daily schedule used in the system;

FIG. 15 is a flow diagram illustrating a weight loss simulation used in the system;

FIG. 16 is an explanatory diagram illustrating an activity intensity calculation procedure in the system; and

FIG. 17 is an explanatory diagram illustrating an input screen of personal detailed data used in the system.

### BEST MODE FOR CARRYING OUT THE INVENTION

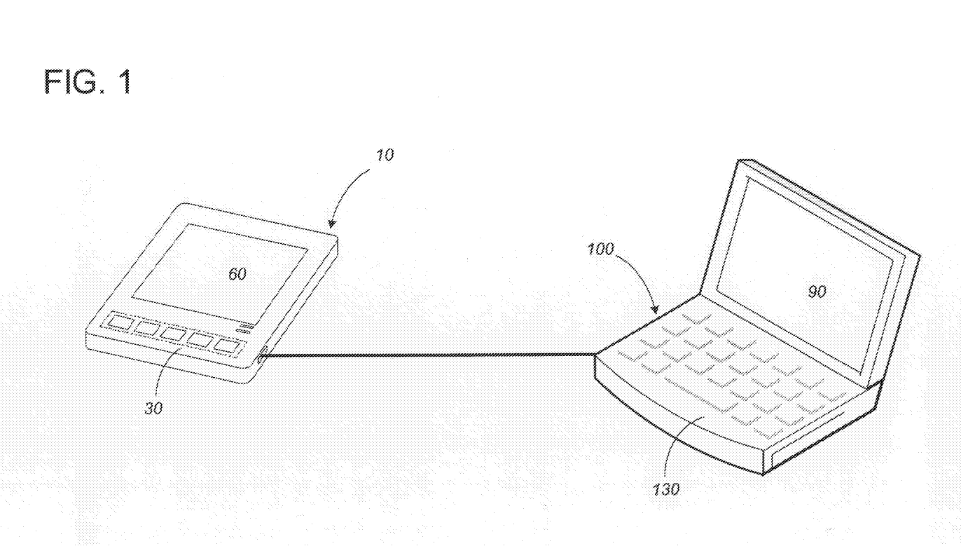

As illustrated in FIG. 1, an activity measurement system according to an embodiment of the present invention comprises a portable terminal 10 for measuring the activity of a user, the portable terminal 10 being designed to measure continuously the activity intensity derived from daily activity and exercise of an user, to analyze the activity trend over a predetermined measurement period, for instance 1 week, and to display the trend; and a server 100 for processing the data acquired by the portable terminal 10. The portable terminal 10 and the server 100 are configured so as to exchange data when connected via a USB cable. The portable terminal 10 has a case provided with a display means 60. The case houses the electronic components that make up an activity detection means. The server 100 comprises a personal computer provided with input means, memory means and display means. The server 100 performs various data analyses, explained below, by executing a dedicated application program.

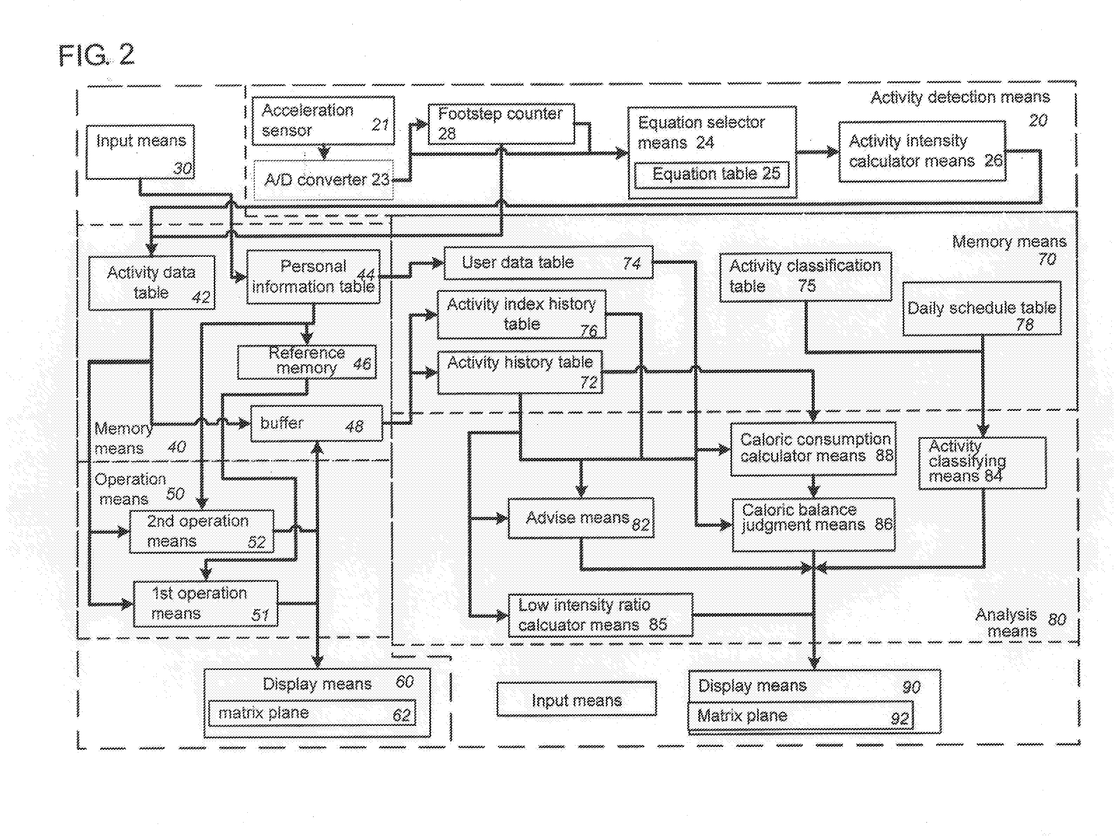

FIG. 2 illustrates various functions of the portable terminal 10 and the server 100. In addition to an input means 30, the portable terminal 10 comprises an activity detection means 20, a memory means 40, an operation means 50, a display means 60 and the input means 30. In addition to an input means 130, the server 100 comprises a memory means 70, an analysis means 80 and a server display means 90.

The portable terminal 10 will be explained first. The activity detection means 20 comprises an acceleration sensor 21 that detects accelerations derived from user activity, an A/D converter 23 that converts an analog output of the acceleration sensor 21 to a digital signal, and an equation selector means 24. The acceleration sensor 21 is configured so as to detect acceleration along three axes x, y and z. The equation selector means 24 extracts accelerations in the three axes, at a sampling frequency of 10 Hz or higher, and obtains a resultant acceleration of the accelerations in each axis. The equation selector means 24 obtains a moving average (V) of the resultant acceleration over a predetermined unit time, for instance over 10 seconds, and obtains an activity intensity (I=MET intensity) using different equations in accordance with the value of the moving average (V). Two threshold values are used for equation selection. To determine an activity intensity that accurately reflects the actual activity, the first threshold value discriminates between walking and routine living activity and the second threshold value discriminates between walking and running. The three equations below, held in an equation table 25, are used as the different equations.

I=a·V+1 [Equation 1]

I=c·V²d·V+e [Equation 2]

I=b·V+1 [Equation 3]

In the equations, a, b, c, d and e are coefficients, wherein a<b; and c, d and e are set to values such that there is continuity between Equation 2 and Equation 3.

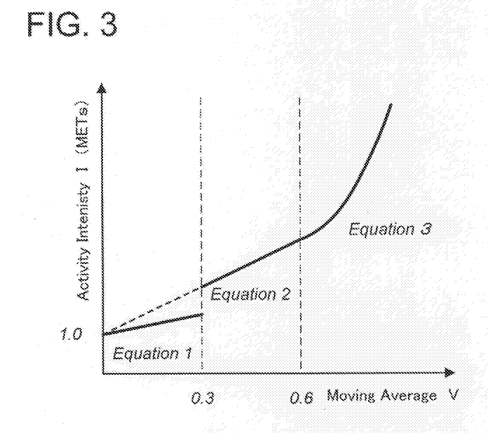

Specifically, Equation 1 is used when the 10-second moving average (V) is equal to or lower than the first threshold value, Equation 2 is used when the moving average (V) is between the first threshold value and the second threshold value, and Equation 3 is used when the moving average (V) exceeds the second threshold value. The first and second threshold values are set for instance to 0.3 and 0.6, as illustrated in FIG. 3, and the relationship between the moving average (V) and the activity intensity (I) is given by the solid lines in the figure.

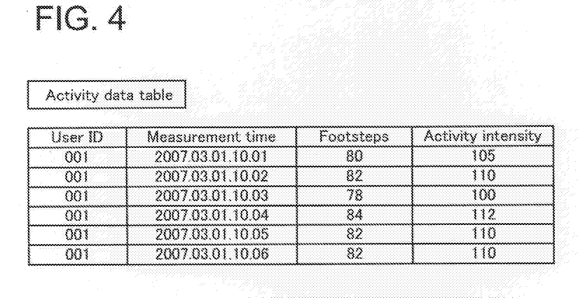

The activity detection means 20 further comprises an activity intensity calculator means 26 and a footstep counter means 28. The activity intensity measurement means 26 calculates activity intensity (METs) every 10 seconds on the basis of a selected equation, and outputs a one-minute average value. The footstep counter means 28 calculates and outputs the number of footsteps per minute, on the basis of the resultant acceleration. The activity intensity and number of footsteps per minute are stored in an activity data table 42, having the data structure illustrated in FIG. 4, that is formed in the memory means 40.

The operation means 50 of the portable terminal 10 comprises a first operation means 51 and a second operation means 52 which calculate an exercise index (EX) indicative of energetic exercise and a daily activity index (DA) indicative of routine living activity within a predetermined measurement time period, for instance one week, on the basis of the number of footsteps and activity intensity read from the activity data table 42, and on the basis also of personal physical characteristics stored in a personal information table 44 and an activity intensity reference held in a reference memory 46 in the memory means 40. The calculation results are displayed on a matrix plane 62 in the display means 60.

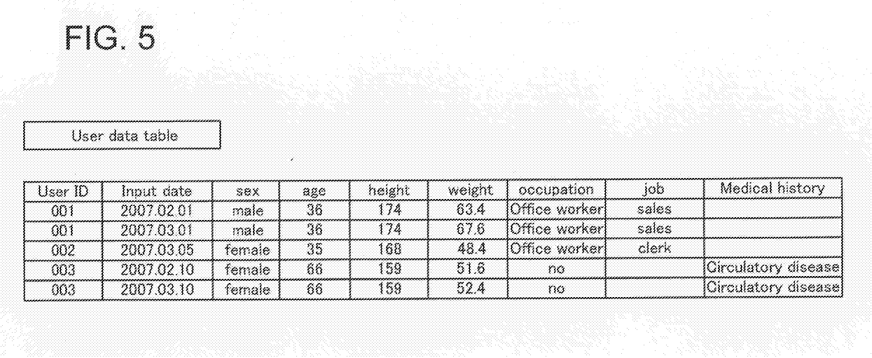

The personal information table 44 stores physical characteristics of the user (sex, age, height, weight) that are inputted by way of the input means 30 of the portable terminal. Upon connection of the portable terminal 10 to the server 100, the data of the personal information table 44 is sent to the user data table 74, having the data structure illustrated in FIG. 5 and provided in the memory means 70 of the server, to update data in the user data table 74. The user data table 74 records, for instance, physical characteristics and other attributes (occupation, job description) relating to a plurality of users in a household. The user data table 74 accumulates data on users that employ a plurality of portable terminals, the data being used for the below-described purposes.

As Table 1 shows, the reference memory 46 holds the above-mentioned activity intensity reference according to user sex and age. The first operation means 51 and the second operation means 52 acquire, from the reference memory 46, an activity intensity reference corresponding to the user sex, age and a below-described personal activity level.

TABLE 1

| | | Age | | | | | |
|---|---|---|---|---|---|---|---|
| | | -29 | 30-39 | 40-49 | 50-59 | 60-69 | 70- |
| Level 1 | Men | 4.7 | 4.4 | 4.3 | 3.7 | 3.6 | 3.3 |
| | Women | 3.9 | 3.9 | 3.7 | 3.7 | 3.7 | 3.4 |
| Level 2 | Men | 5.7 | 5.4 | 5.4 | 5.1 | 4.7 | 4.5 |
| | Women | 4.6 | 4.5 | 4.2 | 4.1 | 4.0 | 3.8 |
| Level 3 | Men | 6.7 | 6.4 | 6.4 | 6.4 | 5.9 | 5.8 |
| | Women | 5.4 | 5.1 | 4.7 | 4.6 | 4.3 | 4.0 |

The first operation means 51 obtains an activity intensity (MH) equal to or greater than the activity intensity reference, for data on the activity intensity per unit time (1 minute) accumulated in the activity data table 42, obtains a total sum of activity intensity (MH) within a measurement time period extending over one week, and obtains the exercise index (EX) as that total sum.

The second operation means 52 obtains a weekly consumption energy (EG) corresponding to activity having a lower activity intensity than the activity intensity reference, as well as a basal metabolic rate (BM) required during the period, on the basis of activity intensity data extending over the immediately preceding week and accumulated in the activity data table 42. The second operation means 52 defines the daily activity index (DA) as the quotient of the consumption energy (EG) divided by the basal metabolic rate (BM). The consumption energy (EG) is determined as the total sum (SKm), over one week, of caloric consumption (Km) per minute, obtained from the equation below, as a function of the basal metabolic rate (Bm) per minute and of an activity intensity (ML) per unit time (1 minute) smaller than the activity intensity reference.

Km=1.2·ML·Bm [Equation 4]

The basal metabolic rate (Bm) per minute is determined as the product (Bm=Bmref×A/60) of a basal metabolic reference per hour BMref (Kcal/m²/hour) and the body surface area A (m²) of the user, determined beforehand in accordance with the sex and age of the user, in a table stored in the reference memory 46, as shown in the table below.

TABLE 2

| | Age | | | | | | | | | | | |
|---|---|---|---|---|---|---|---|---|---|---|---|---|
| | 17 | 18 | 19 | 20-29 | 30-39 | 40-49 | 50-59 | 60-64 | 65-69 | 70-74 | 75-79 | 80- |
| Men | 40.3 | 39.6 | 38.8 | 37.5 | 36.5 | 35.6 | 34.8 | 34.0 | 33.3 | 32.6 | 31.9 | 30.7 |
| Women | 36.0 | 35.6 | 35.1 | 34.3 | 33.2 | 32.5 | 32.0 | 31.6 | 31.4 | 31.1 | 30.9 | 30.0 |

The body surface area A (m²) is obtained as a function of body weight and height, using for instance the equation below.

A=0.008883·W^{0.444}·H^{0.663} [Equation 5]

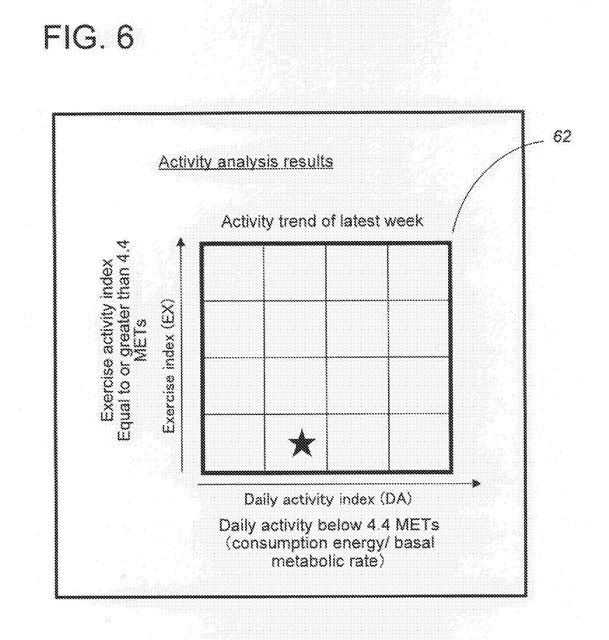

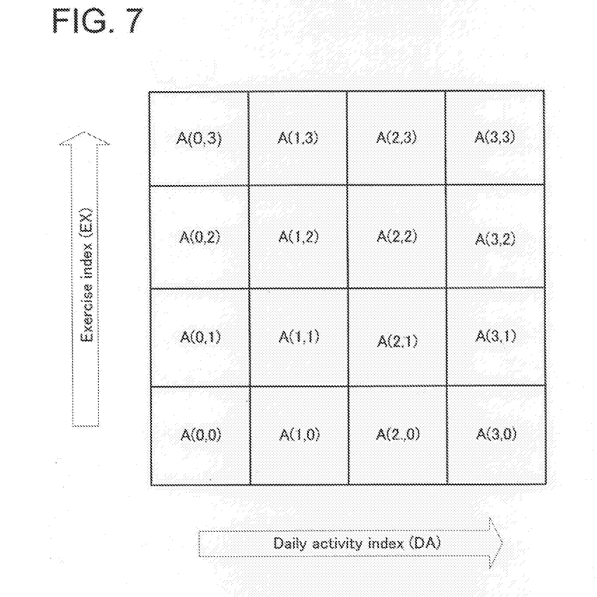

The exercise index (EX) and daily activity index (DA) obtained as described above are stored in a buffer 48 of the memory means 40 and are displayed on the X-Y matrix plane 62 in a display, as illustrated in FIG. 6. The exercise index (EX) and the daily activity index (DA) are divided each into four ranges (0-3), as illustrated in FIG. 7. Within a 4×4 matrix, the position of an activity element [A(x, y)], which is a combination of exercise index (EX) and daily activity index (DA), is indicated by a star sign (shown in FIG. 6). As a result, the activity trend of the user can be grasped at a glance by displaying thus the amount of exercise derived from energetic exercise and the amount of exercise derived from routine living activity.

As Table 1 shows, the activity intensity reference used by the first operation means 51 and the second operation means 52 has three pre-established activity levels. Each level is set so as to correspond to the daily activity index (DA) obtained as described above, so that each level is automatically selected depending on the average daily activity index (DA) obtained for, for instance, one week or one month. Accordingly, the first operation means 51 and the second operation means 52 are configured so as to read a daily activity index (DA) extending over an immediately previous predetermined period stored in the buffer 48, upon selection of an activity intensity reference, and to select, from a table in the reference memory 46, an activity intensity reference suited to the activity of the user. An exercise index (EX) and daily activity index (DA) that best reflect the activity level of the user can be obtained as a result. The activity intensity reference is set assuming that the activity level of the user, i.e. the daily activity index (DA), increases from level 1 through level 2 to level 3.

Preferably, the levels corresponding to the activity intensity reference selected by the first operation means 51 and the second selection means 52 are also displayed on the display means 60. In this case, the data of the respective level is recorded in the buffer 48 and is provided to the display means 60.

The display means 60 of the portable terminal 10 is configured in such a manner so as to have an intensity display mode for displaying activity intensity (METs) calculated per unit time (1 minute), in addition to an analysis mode in which there is displayed an activity trend, on the above-described matrix plane 62. A short-time analysis mode is also set for providing, on the matrix plane, a daily activity trend wherein the measurement time period is one day. A mode selection button provided in the input means 30 of the portable terminal 10 allows switching between these display modes.

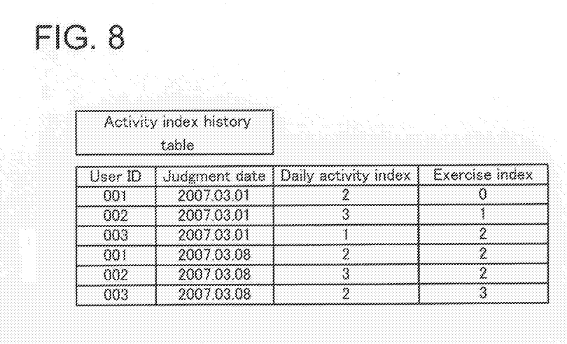

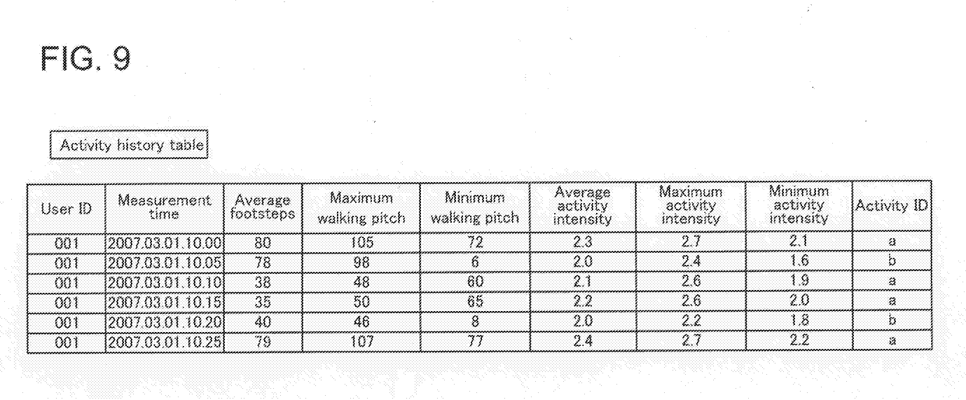

When the portable terminal 10 is connected to the server 100, the exercise index (EX) and daily activity index (DA) stored in the buffer 48, as well as the data in the activity data table 42, are sent to, and stored in, the activity index history table 76 and the activity history table 72 of the server 100. The activity index history table 76 has a data structure such as the one illustrated in FIG. 8, and the activity history table has the data structure illustrated in FIG. 9. Each table stores collectively data on different users, these data being then used for detailed analysis on a respective different user.

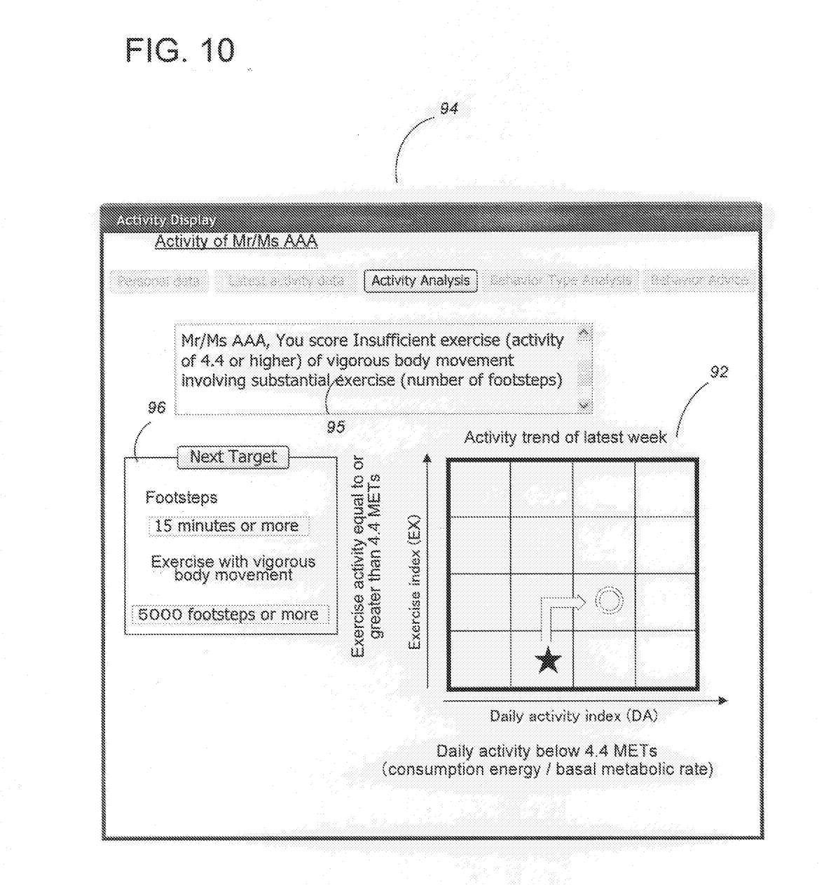

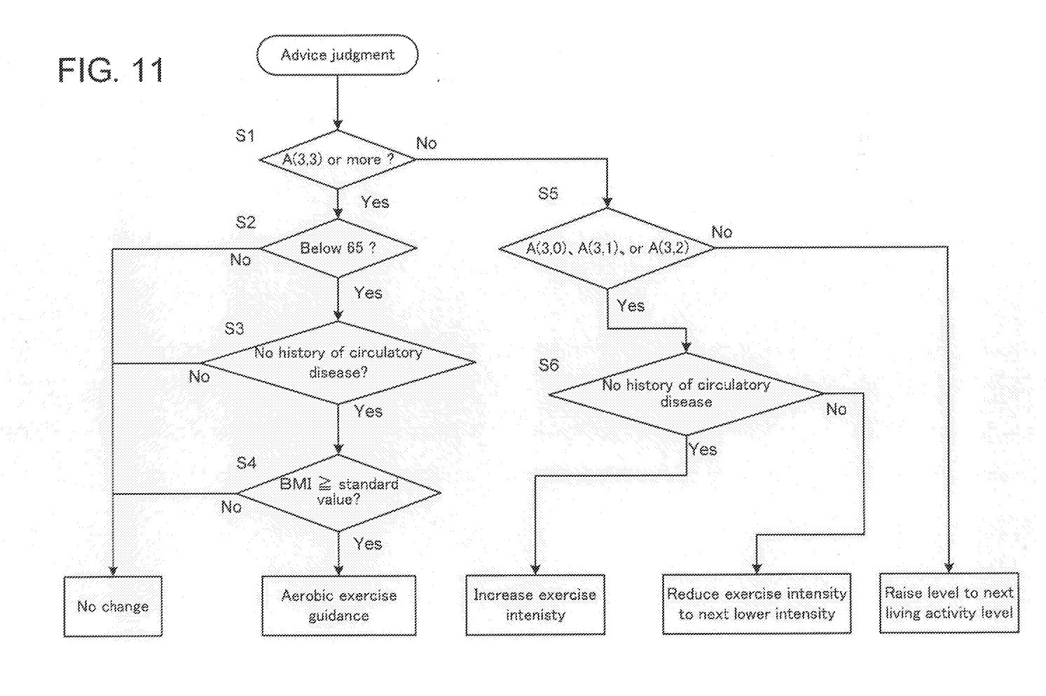

The server 100 is provided with an analysis means 80 that comprises, as illustrated in FIG. 2, an advise means 82 as well as a below-described activity classifying means 84, a caloric balance judgment means 86, a caloric consumption calculator means 88, and a low intensity ratio calculator means 85. The advise means 82 is provided for rendering advice on future exercise to the user. The advise means 82 extracts the latest data for a specific user, from the activity index history table 76 and user data table 74, and displays the advice content in text boxes 95, 96, within a window form 94 provided in the server display means 90, in a matrix plane 92 identical to the above-described one, as illustrated in FIG. 10. The advice content is a combination of specific numerical values and types of exercise that are stored beforehand in the advise means 82. The advice content is determined on the basis of the scheme illustrated in the flowchart of FIG. 11. In this scheme, it is judged first whether an activity element A (x,y) of the exercise index (EX) and the daily activity index (DA), acquired from the activity index history table 76, is equal to or greater than a maximum (A(3,3)) (step S1). If this condition is satisfied, it is judged whether the age is below 65 years (S2). If the age is not below 65, the advice content is set to “no change”. The advice content is likewise set to “no change” if in S3 it is judged that there is a history of circulatory disease, or in the absence of circulatory disease, if it is judged in S4 that the BMI (weight/height²) is not equal to or greater than a standard value. When in S4 it is judged that the BMI is equal to or greater than a standard value, the advice content is decided to the effect of urging aerobic exercise such as cycling or jogging. When in S1 it is judged that the activity element A (x,y) is lower than A(3,3), the process proceeds to S5, where it is judged whether the activity element A (x,y) coincides with A(3, 0), A(3, 1) or A(3, 2). If this condition is not satisfied, the advice content is set to “raise level to next living activity level”. When the condition of S5 is satisfied, it is judged in S6 whether there is a history of circulatory disease. If there is no circulatory disease, the advice content is set to “increase exercise intensity”. Else, the advice content is set to “reduce exercise intensity to next lower intensity”.

The advise means 82 decides the optimal activity element A(x,y) on the basis of the advice content determined as described above, and displays a target mark (\textcircled{×}) at a corresponding coordinate position on the matrix plane 92, as illustrated in FIG. 10, together with an arrow that indicates the optimal route to reach the target activity element. The advise means 82 can also be configured so as to further display, in the form illustrated in FIG. 10, an activity level judged to be an activity level (given in Table 1) corresponding to the personal daily activity index (DA) measured over a predetermined period of time as described above. In particular, an exercise intensity value corresponding to the determined activity level can be added to the advice content.

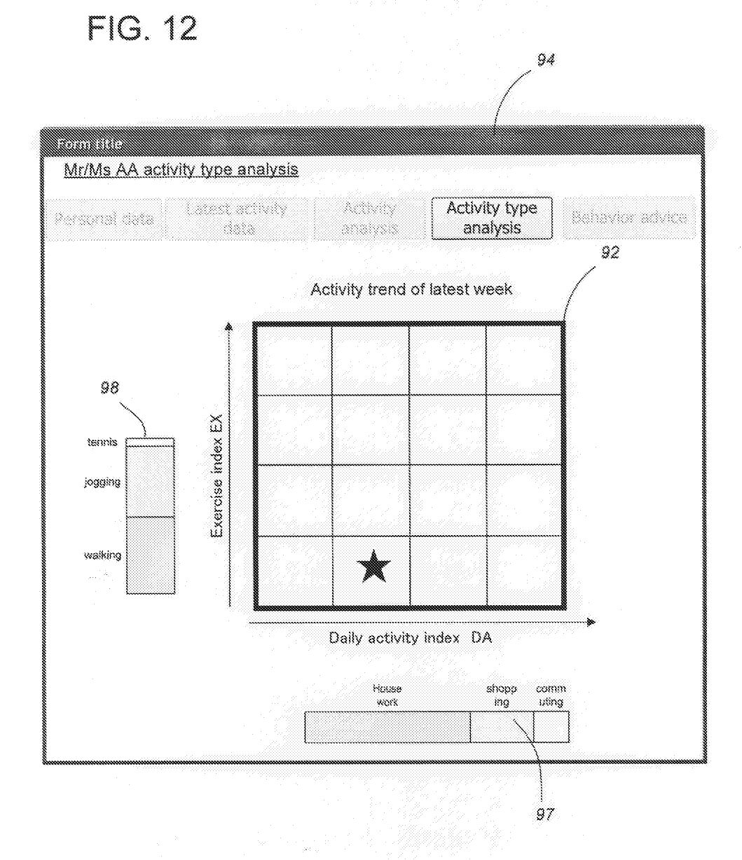

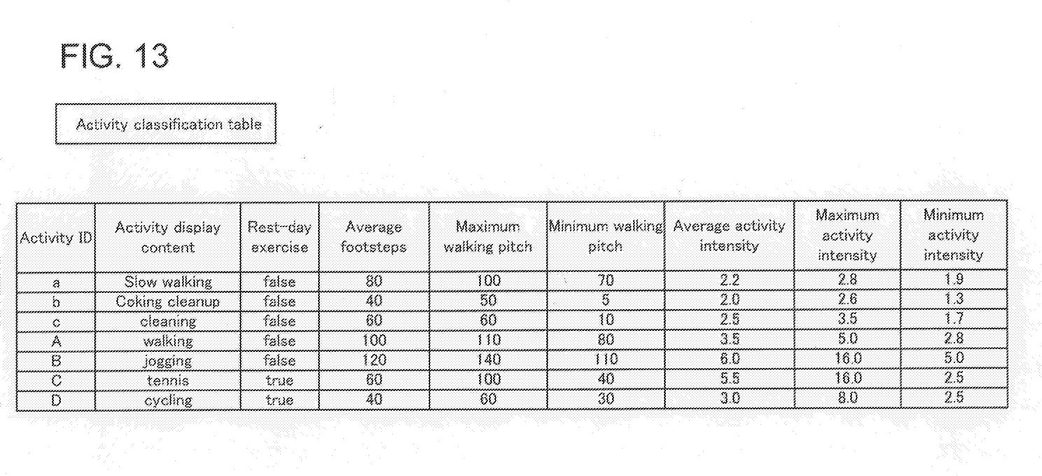

On the basis of the data accumulated in the activity history table 72, the activity classification judgment means 84 of the analysis means 80 determines the type of exercise and living activity of the latest one-week period, which is the predetermined measurement time period, and causes the type and ratio thereof to be displayed as bar graphs 97, 98 in a window form where the exercise index (EX) and the daily activity index (DA) are contrasted, as illustrated in FIG. 12. The type of exercise and the type of routine living activity are defined mapped to parameters that include average number of footsteps, minimum/maximum walking pitch, average activity intensity and minimum/maximum activity intensity, in an activity classification table 75 having the data structure illustrated in FIG. 13 and provided in the memory means 70. Every 5 minutes, the activity classification judgment means 84 extracts the same parameters, from the time-series data recorded in the activity history table 72 illustrated in FIG. 9, and specifies, every 5 minutes, the activity type that these parameters resemble, by way of a cluster analysis. When the specified activity type exhibits continuously a predetermined pattern over a predetermined judgment time, for instance 25 minutes, the activity type is judged to be the activity type within this predetermined judgment time. For instance, when an activity ID (a) denoting an exercise type (“slow walking”), and an activity ID (b) denoting “cooking/cleanup” succeed each other as “a, b, a, a, b, a” over 25 minutes, the pattern “a, b, a” is judged to be “a, a, a”. The exercise within this judgment time period is judged to be “slow walking”. Thus, activity types in 25-minute units are compiled within a measurement time period of one week, to determine the types of activity and ratios thereof.

Herein, the types of activity (slow walking, cooking/cleanup, cleaning and the like) and the ratios thereof, obtained on the basis of the activity intensity indicative of routine living activity, i.e. an activity intensity smaller than an activity reference specified by the user, are displayed as the bar graph 97 along the daily activity index (DA) in the X-axis of the matrix plane 92 within the window form 94 illustrated in FIG. 12. Meanwhile, the types of activity (walking, jogging, tennis, cycling) determined on the basis of the activity intensity indicative of energetic exercise, and the activity type ratios, are displayed as the bar graph 98 along the exercise index (EX) in the Y-axis.

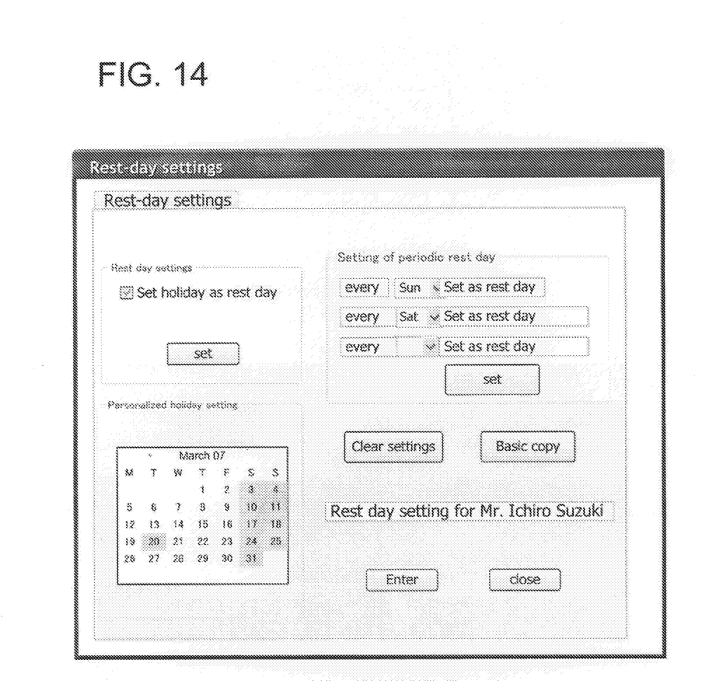

The activity classifying means 84 looks up a daily schedule table 78 set in the memory means 70, in such a manner so as to disregard the activity type if the activity type, judged every day within the measurement time period, is an activity type not planned for that day. For instance, if the activity type is judged to be tennis or jogging during work hours on weekdays, the activity type is disregarded, and there is used an activity type judged to be the next best activity type. The daily schedule table 78 is created by filling a rest-day setting form, illustrated in FIG. 14, that is displayed on the display means 90. The activity classification table 75 has a field that decides whether an activity is exercise planned for a rest day. The relationship between rest days and activity types is established based on user input.

The low intensity ratio calculator means 85 is provided in the analysis means 80 in order to display the ratio of time during which low-intensity activity is carried out within a measurement time period of one day. The low intensity ratio calculator means 85 acquires the number of footsteps and activity intensity per minute from the activity history table 72, obtains the proportion of time, within 24 hours, during which the number of footsteps per minute is equal to or lower than 80 and during which the activity intensity is smaller than the above-described activity reference, and displays the obtained proportion on the display means 90. The measurement time period above is not only set to one day, and may be set to one week or one month. In the present embodiment, the analysis means 80 is provided in the server 100. However, the analysis means 80 may also be provided in the portable terminal 10, so that the low-activity time ratio is displayed on the display means 60 of the portable terminal 10.

The caloric balance judgment means 86 in the analysis means 80 is provided in order to obtain changes in the user body fat and the variation of caloric consumption for each day over a predetermined period of time, for instance one month, and to display graphs of the variations on the display means 90. The caloric consumption per day, which is calculated by the caloric consumption calculator means 88, is obtained from the caloric consumption per minute that is worked out using equation 4 on the basis of the basal metabolic rate and the activity intensity specific to the user, as is the case in the above-described second operation means 52. The change in body fat is determined on the basis of age, sex, height and weight, which constitutes the body fat determination data, as stored in the user data table 74.

The variation in caloric consumption and body fat is obtained for each day based on the criteria given in the table below. The diet amount is determined at the same time by combining the above variations. In the graphs displayed on the display means 90 there can also be used values of actual caloric consumption and body weight, instead of a (+)(−) display.

TABLE 3

| Caloric | | |
|---|---|---|
| consumption | Body weight | Diet amount |
| variation | variation | judgment |
| + | + | 2+ |
| | 0 | + |
| | − | 0 |
| 0 | + | + |
| | 0 | 0 |
| | − | + |
| − | + | 0 |
| | 0 | − |
| | − | 2− |

Specifically, caloric consumption and body fat are compared with those of a previous date, and when the increase or decrease exceeds an increment or decrement (ΔE, ΔW) of a predetermined value, the caloric balance judgment means 86 performs a respective (+) (−) judgment and estimates the dietary intake at the time by combining the increments/decrements for the given time. The results of the dietary intake judgment are also displayed as a graph on the display means 90. This allows drawing the attention of the user towards the dietary intake.

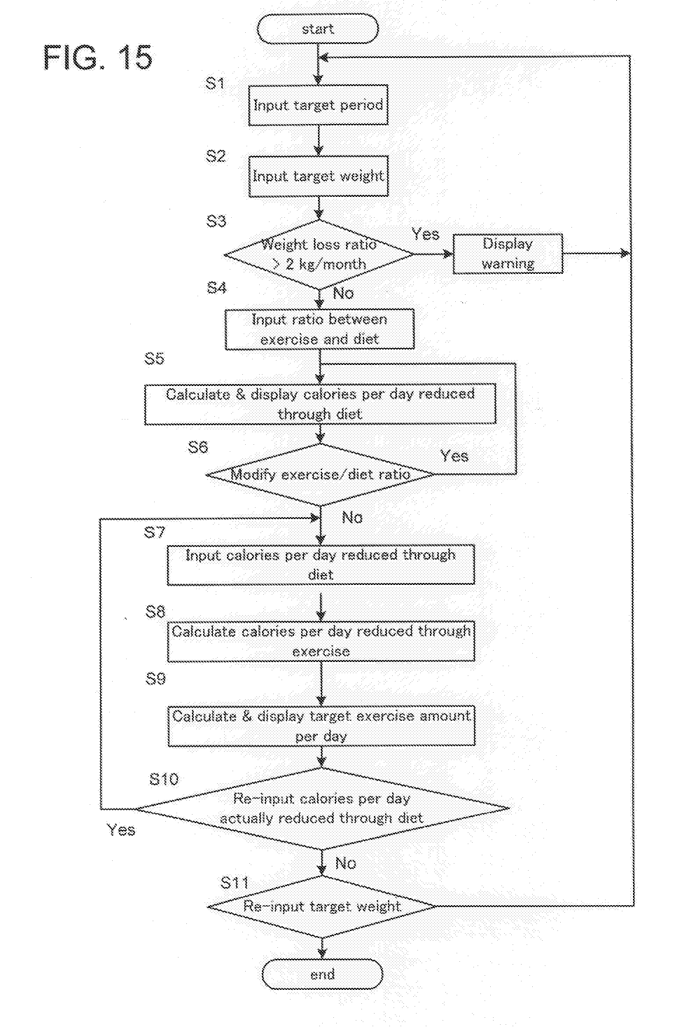

The caloric balance judgment means 86 has a supplementary weight loss simulation function for rendering advice to the user on appropriate dietary limits and therapeutic exercise. The weight loss simulation is performed by executing the program illustrated in the flowchart of FIG. 15, on the basis of “target period”, “target body weight”, “exercise/diet ratio” and “weight-loss energy through dietary restriction” inputted as input items prompted by the display means 90. Firstly, the target period and the target body weight are inputted in steps S1 and S2. The target period default is set to 3 months. The default target body weight is the set to the current body weight. The user can modify these target values arbitrarily. In step S3 there is calculated the weight loss rate per month, on the basis of the target period and the target body weight. When the weight loss rate exceeds a predetermined value (for instance, 2 kg/month), a warning is displayed, and the process returns to S1. In step S4 there is prompted input of a ratio of calorie reduction through exercise and diet. The exercise/diet ratio is a ratio that distributes, between exercise and diet, the daily caloric consumption necessary for reducing the current body weight (Wb) down to the target body weight (Wa). A value apportioning 30% of the caloric consumption to diet is set as the default value, which can be selected within a range from 10% to 90%. In S5 there are calculated and displayed the calories per day decreased through diet, on the basis of the ratio inputted in S4. In S6 the exercise/diet ratio can be modified in the light of the calories calculated in S5. If no modifications are necessary, the program returns to S5. In S7 there is prompted input of caloric weight loss through actual diet reduction by the user, in the light of the calories per day to be reduced through diet as displayed in S5. In S8 there is calculated the caloric weight loss through exercise that offsets the caloric weight loss inputted in S7. Next, in S9, there is calculated and displayed the amount of daily exercise to be carried out in order to achieve the caloric weight loss through exercise calculated in 38. As the amount of exercise there is calculated the necessary activity time for, for instance, “ordinary walking” in which the activity intensity is represented by 3 METs, “brisk walking” by 4 METs, and “cycling” by 3 METs. The type of exercise is displayed alongside the respective activity time. Next, in S10 the user is allowed to modify the calories per day to be reduced through actual diet, by referring to the display of S9. When there is a modification input, the process returns to S7. If there is no modification, the process proceeds to S11, where the target body weight can be modified. If there is no modification, the process ends, or returns to S1 if there is a modification. Other than body weight, the amount of visceral fat, abdominal circumference or BMI (weight/height²), or a combination of the foregoing, can also be used as the body fat judgment data.

In the above embodiment, the portable terminal 10 is provided with an equation selector means 24 that selectively uses different equations in order to obtain an activity in accordance with the characteristics of the user. In the above explanation, an equation is selected from among the plurality thereof in accordance with the acceleration measured at the portable terminal, as illustrated in FIG. 3. However, the present invention is not necessarily limited thereto, and the equations can also be selected on the basis of the number of footsteps detected by the footstep counter means 28 of the portable terminal 10, or based on a combination of number of footsteps and acceleration. For instance, when using 60 as the first threshold value, 120 as the second threshold value, and less than 60 as the number of footsteps per minute, the equation selector means 24 selects the equation represented by Equation 1 above, the detected activity being assumed to derive from living activity. When the number of footsteps per minute ranges from 60 to less than 120, the detected activity is assumed to derive from walking and Equation 2 is used. When the number of footsteps per minute is 120 or greater, the detected activity is assumed to derive from running and Equation 3 is used.

When using the number of footsteps and acceleration for equation selection, the equation selector means 24 acquires from the equation table 25 an equation corresponding to the acceleration, and an equation corresponding to the number of footsteps obtained by the footstep counter means 28. When these two equations are identical, that identical equation is used. When the equations do not coincide, equation use is prohibited, and activity intensity is deemed to be not calculable. When the equations do not coincide, the detection precision of user activity is enhanced by deeming that calculation of activity intensity is unfeasible under circumstances where the portable terminal, unattached to the body, is acted upon by external forces, for instance when moved with the hands.

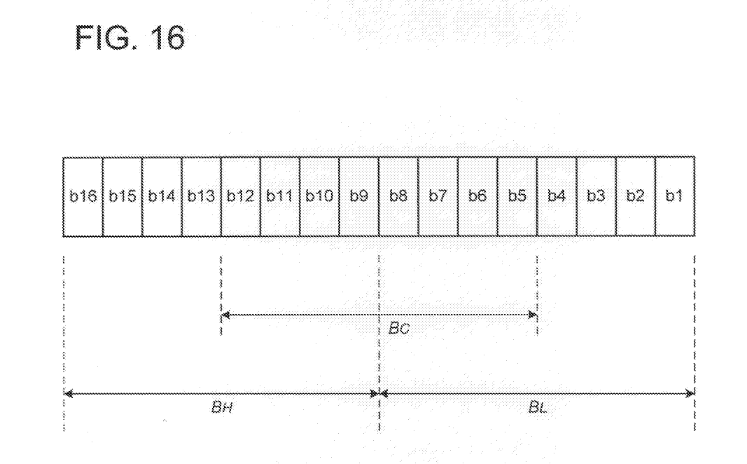

The activity intensity calculator means 26 is configured in such a manner so as to calculate activity intensity per unit time (10 seconds) by using the above equations on the basis of a 16-bit acceleration outputted by an A/D converter. One of the three partial bit series (8-bit long) from among the 16-bit series illustrated in FIG. 16 is selectively used, in accordance with the magnitude of acceleration and/or the number of footsteps, in order to increase the speed of the computation process. Specifically, when the acceleration and the number of footsteps are smaller than the first threshold value there is used a lower bit series BL (b1 to b8). When the acceleration and the number of footsteps lie between the first threshold value and the second threshold value there is used a central bit series BC (b5 to b12). When the acceleration and the number of footsteps are equal to or greater than the second threshold value there is used a higher bit series BH (b9 to b16).

The above-described embodiment was illustrated by way of an example in which the first operation means 51 and the second operation means 52 that calculate the daily activity index (DA) and the exercise index (EX) are provided in the portable terminal 10. However, the present invention is not necessarily limited to such an embodiment. The first operation means, the second operation means and the reference memory 46 that stores different equations for obtaining the activity intensity depending on the user may also be provided in the analysis means controller 80 of the server 100. When the portable terminal 10 is connected to the server 100, the exercise index (EX) and the daily activity index (DA) obtained by the server are transmitted in this case to the portable terminal 10, and the results are displayed in the matrix plane 62 of the display means 60.

Alternatively, the memory means 70 and analysis means 80 of the server 100 may be provided in the portable terminal 10. This allows configuring a self-contained portable terminal having all the functions of the systems of the present invention.

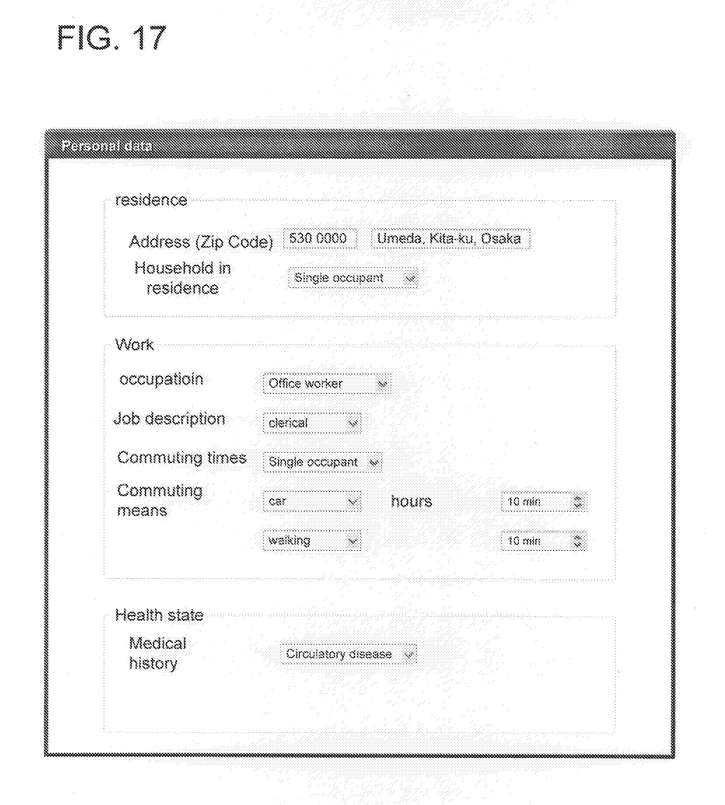

The above-described server 100, moreover, can be set so as to be capable of communicating with other data servers (not shown) in a web. In this case, the data server is configured so as to compile the records of the activity history table 72, the activity index history table 76 and the user data table 74 of the server 100 belonging to each user. The data server is set so as to transmit the exercise index (EX) and daily activity index (DA) relating to another person in response to a request from a respective server 100. The advise means 82 of each server 100 may be supplemented with a reference function for referring to the activity trend of another person belonging to the same user category as the owner of the server 100. Upon selection of this reference function, a reference request with a code that specifies the user is sent to the data server. Upon reception of the reference request, the data server performs a cluster analysis to specify a user, belonging to the same user category as the user of the transmission source, and having similar sex, age, height, weight, occupation, job description, medical history and other detailed parameters. The data server sends then the compiled exercise index (EX) and daily activity index (DA) of the specified similar user to the transmission-source server 100. As a result, the exercise index (EX) and daily activity index (DA) of another person are also displayed, for reference, on the matrix plane of the display means of the transmission-source server 100. Details on personal data of the user are inputted using an input form, for instance such as the one illustrated in FIG. 17, provided by a server display means 90 of each server 100. These details are accumulated in the data server, to be used for user classification.

1. An activity measurement system comprising:
an activity detection means configured to detect an activity of a user and obtain an activity intensity per unit time with regard to the activity;
a first operation means configured to obtain a first index which is a product of the activity intensity exceeding a predetermined reference intensity and a time during which the activity intensity exceeds said reference intensity within a predetermined measurement time period;
a personal information table configured to store physical characteristics of the user;
a second operation means configured to provide a second index different from said first index based upon said activity intensity; and
a display means including a display providing a X-Y matrix plane and being configured to show an activity information indication which is a combination of said first and second indexes at a position in an coordinate system defined by said fist and second indexes;
wherein said second operation means is configured to obtain a total of a consumption energy defined by a function of the activity intensity per unit time and the physical characteristics of the user read out from said personal information table within said measurement time period, to obtain, based on the physical characteristics of the user read out from said personal information table, a basal metabolic rate required within said measurement time period, and to provide said second index which is the consumption energy divided by said basal metabolic rate.

2. An activity measurement system as set forth in claim 1, wherein
said second operational means is configured to obtain said consumption energy with reference to the activity intensity only below said reference intensity.

3. An activity measurement system as set forth in claim 1, wherein
said measurement time period is defined to be one week.

4. An activity measurement system as set forth in claim 2, further including:
a reference memory configured to store said reference intensity as associated with the age and sex of the user,
said second operational means being configured to select the reference intensity according to the age and sex of the user.

5. An activity measurement system as set forth in claim 1, further including:
a terminal case adapted in use to be carried by the user,
said terminal case being provided with said activity detection means, said first operation means, said second operation means, and said display means.

6. An activity measurement system as set forth in claim 1, wherein
said system includes a portable terminal adapted in use to be carried by the user, and a server configured to transmit and receive data to and from said portable terminal,
said portable terminal being provided with said activity detection means, and
said server being provided with said first operation means, said second operation means, and said display means.

7. An activity measurement system as set forth in claim 1, wherein
said system includes a plurality of portable terminals each adapted in use to be carried by the user, and a server configured to transmit and receive data to and from said portable terminals,
said portable terminal being provided with said activity detection means, said first operation means, said second operation means, and said display means,
said server being configured to have an activity history table that stores said first index and said second index obtained by each of said portable terminals, and a user data table that stores attributes of the users;
said server being provided with a server side display means having a X-Y matrix plane, said server side display means being configured to provide the activity information indication transmitted from a particular one of said portable terminals, as well as to provide the activity information indication given for the other users classified in the same user group, said activity information indication being displayed in said X-Y matrix.

8. An activity measurement system as set forth in claim 2, wherein
said activity detection means includes an acceleration sensor, an activity intensity calculation means, and a footstep counter means,
said acceleration sensor being configured to output acceleration data generated from the activity of the user,
said activity intensity calculation means being configured to determine, based upon said acceleration data, said activity intensity per unit time,
said footstep counter means being configured to count the number of footsteps per unit time based upon the acceleration data,
said system further including:
an activity history table configured to store a time series data of the activity intensity and the number of the footsteps per unit time in association with a measurement time;
an activity classification table configured to classify kinds of the activity in terms of the activity intensity and the number of footsteps as parameters; and
an activity classifying means which is configured to analyze the time-series data of the activity intensity and the number of footsteps at each measurement time and compare the same from those of the activity classification table so as to determine similarity of said data with one of the kinds of the activity in said activity classification table, to identify the kind of the activity at said each measurement time based upon the similarity, and to obtain a ratio of each identified kind of the activity within the measurement time period,
said display means being configured to show the ratio of the kind of the activity together with a content indicative of the kind of the activity along the coordinate axes of said matrix plane.

9. An activity measurement system as set forth in claim 7, wherein
said activity classifying means is configured to identify the kind of the activity by means of the cluster analysis.

10. An activity measurement system as set forth in claim 8, wherein
said time series data includes an average activity intensity, a maximum activity intensity, minimum activity intensity, an average number of footsteps, a maximum foot pitch, and a minimum foot pitch.

11. An activity measurement system as set forth in claim 3, further including:
a daily schedule table configured to hold a daily behavior schedule of the user,
said activity classifying means being configured to judge whether or not the kind of the activity obtained for each day within the measurement time period is in match with the kind of the activity expected in the daily behavior schedule corresponding to that day, and ignore the kind of the activity not expected in that day.

12. An activity measurement system as set forth in claim 1, wherein
said activity detection means comprises an acceleration sensor, an activity intensity calculator means, and an equation selector means,
said acceleration sensor being configured to output acceleration data generated by the activity of the user,
said activity intensity calculator means being configured to obtain said activity intensity by use of a particular equation which is a function of said acceleration data,
said equation selector means having an equation table holding a plurality of different equations respectively associated with the different acceleration data, and being configured to retrieve, from said equation table, the equation corresponding to the acceleration data detected at said acceleration sensor, and provide the retrieved equation to said activity intensity calculator means.

13. An activity measurement system as set forth in claim 1, wherein
said activity detection means comprises an acceleration sensor, an activity intensity calculator means, a footstep counter means, and an equation selector means,
said acceleration sensor being configured to output acceleration data generated by the activity of the user,
said activity intensity calculator means being configured to obtain said activity intensity by use of a particular equation which is a function of said acceleration data,
said footstep counter means being configured to determine the number of footsteps per unit time from said acceleration data,
said equation selector means having an equation table holding a plurality of different equations respectively associated with the different number of the footsteps, and being configured to retrieve, from said equation table, the equation corresponding to the number of the footsteps detected at said footstep counter means, and provide the retrieved equation to said activity intensity calculator means.

14. An activity measurement system as set forth in claim 1, wherein
said activity detection means comprises an acceleration sensor, an activity intensity calculator means, a footstep counter means, and an equation selector means,
said acceleration sensor being configured to output acceleration data generated by the activity of the user,
said activity intensity calculator means being configured to obtain said activity intensity by use of a particular equation which is a function of said acceleration data,
said footstep counter means being configured to determine the number of footsteps per unit time from said acceleration data,
said equation selector means having an equation table holding a plurality of different equations respectively associated with the different number of the footsteps, and also with the different acceleration data,
said equation selector means being configured to retrieve, from said equation table, the equation corresponding to the number of the footsteps detected at said footstep counter means as
well as to the acceleration data detected at said acceleration sensor, and provide the retrieved equation to said activity intensity calculator means only when such equation is found to correspond to the number of the footsteps and at the same time to the acceleration data.

15. An activity measurement system as set forth in claim 12, wherein
said activity detection means includes an A/D converter configured to convert the output from said acceleration sensor into a digital data defined by a predetermined bit array,
said activity intensity calculator means being configured to assign the different equations to different partial bit series which are different from each other within said digital data, to extract, from said digital data, the partial bit series corresponding to the equation selected at said equation selector means, and to calculate the activity intensity by use of a numerical value expressed by said partial bit series.

16. An activity measurement system as set forth in claim 1, wherein
said activity detection means comprises an acceleration sensor, an activity intensity calculator means, and a footstep counter means,
said acceleration sensor being configured to output a time series of acceleration data generated by the activity of the user,
said activity intensity calculator means being configured to obtain, from the time series acceleration data, said activity intensity at predetermined intervals by use of a particular equation,
said footstep counter means being configured to determine, from said time series of the acceleration data, the number of footsteps per predetermined unit time,
said activity measurement system further including a low intensity ratio calculator means configured to obtain, within said predetermined measurement time period, a low exercise time period in which the number of footsteps is below a predetermined reference and at the same time said activity intensity is within a predetermined reference range, and to provide a ratio of said low exercise time period to said measurement time period, allowing said display means to display said ratio.

17. An activity measurement system as set forth in claim 1, wherein
said activity detection means comprises an acceleration sensor, and an activity intensity calculator means,
said acceleration sensor being configured to output a time series of acceleration data generated by the activity of the user,
said activity intensity calculator means being configured to obtain, from the time series acceleration data, said activity intensity at predetermined intervals by use of a particular equation,
said activity measurement system further including a user table, a caloric consumption calculator means, and a caloric balance judgment means,
said user table holding records of individual information including age, physical characteristics, and fat judgment data specific to the user,
said caloric consumption calculator means being configured to obtain a basal metabolic rate based on the age and the physical characteristics, and calculate a caloric consumption based upon thus obtained basal metabolic rate and the activity intensity within a predetermined judgment time period,
said caloric balance judgment means being configured to obtain a variation of the caloric consumption as well as a variation of said fat judgment data within the judgment time period so as to give a judgment result in terms of a combination of the variations, and
said display means being configured to display said judgment result.

18. An activity measurement system as set forth in claim 17, wherein
said fat judgment data includes at least one of body weight, visceral fat amount, abdominal circumference, and BMI (weight/height).

19. An activity measurement system as set forth in claim 13, wherein
said activity detection means includes an A/D converter configured to convert the output from said acceleration sensor into a digital data defined by a predetermined bit array,
said activity intensity calculator means being configured to assign the different equations to different partial bit series which are different from each other within said digital data, to extract, from said digital data, the partial bit series corresponding to the equation selected at said equation selector means, and to calculate the activity intensity by use of a numerical value expressed by said partial bit series.
